(19) United States
(12) Patent Application Publication
(10) Pub. No.: US 2024/0145035 A1
(43) Pub. Date: May 2, 2024

(54) ANALYZING PER-CELL CO-EXPRESSION OF CELLULAR CONSTITUENTS

(71) Applicant: BioLegend, Inc., San Diego, CA (US)

(72) Inventors: Santosh Putta, Foster City, CA (US); Nikil Wale, Millbrae, CA (US); Wesley Jensen, San Francisco, CA (US); Srikar Devakonda, Cupertino, CA (US)

(21) Appl. No.: 18/497,763

(22) Filed: Oct. 30, 2023

Related U.S. Application Data
(60) Provisional application No. 63/381,819, filed on Nov. 1, 2022.

(51) Int. Cl. G16B 25/10; G16B 40/20; G16B 45/00

(52) U.S. Cl. CPC G16B 25/10; G16B 40/20; G16B 45/00

(57) ABSTRACT

A data structure relating to a sample of cells is described. The data structure includes first data elements each representing one of a number of first-degree nodes. Each of the first-degree nodes corresponds to a different one of a number of cellular constituents. Each first data element includes a quantitative indication of the portion of cells of the sample in which the constituent has positive expression. The data structure also includes second data elements each representing one of a number of greater-than-first-degree nodes, which each correspond to a different subset of the constituents of size two or more. Each second data element includes a quantitative indication of the portion of cells of the sample in which the subset of constituents all have positive expression. The contents of the data structure are usable to generate a visual co-expression graph characterizing the sample.

### CROSS-REFERENCE TO RELATED APPLICATIONS

This Application claims the benefit of U.S. Provisional Application No. 63/381,819 filed Nov. 1, 2022 and entitled “ANALYZING PER-CELL CO-EXPRESSION OF CELLULAR CONSTITUENTS,” which is hereby incorporated by reference in its entirety.

In cases where the present application conflicts with a document incorporated by reference, the present application controls.

### BACKGROUND

Single-cell analysis techniques enable measuring the expression level of different constituents within individual cells, including constituents such as proteins and RNA transcripts. For example, flow cytometry instruments pass single cells through the path of a laser, and interrogate them with various visible and fluorescent light sources that allow assessment of protein composition; mass cytometry instruments apply heavy metal ion tags as labels in place of fluorochromes, and read them using time-of-flight spectrometry; and Cellular Indexing of Transcriptomes and Epitopes by Sequencing (“CITE-Seq”) approaches use DNA-barcoded antibodies to detect proteins.

A common use of these single-cell analysis techniques involves collecting a sample of cells from a particular subject; using an instrument to apply one of the analysis techniques to each cell to obtain an expression level for each of one or more constituents; and outputting a table identifying the measured expression level of each constituent in each cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram showing some of the components typically incorporated in at least some of the computer systems and other devices on which the facility operates.

FIG. 2 is a data flow diagram showing operation of the facility in some embodiments.

FIG. 3 is a flow diagram showing a process performed by the facility in some embodiments to generate a constituent per-cell co-occurrence graph for a sample of cells.

FIG. 4 is a table diagram showing sample contents of an instrument output table used by the facility in some embodiments to store instrument output data for a single sample.

FIG. 5 is a histogram with sample contents showing the level of expression of CD4+ T Cells in the sample of the example.

FIG. 6 is a plot diagram showing a joint expression distribution for two of the constituents in the data for the example.

FIG. 7 is a table diagram showing sample contents of a constituent co-expression table used by the facility in some embodiments to store the constituent co-expression count for each cell type and constituent subset of a sample, on which a co-expression graph can be based.

FIG. 8 shows a sample cumulative co-expression graph generated by the facility for the example sample.

FIG. 9 is a sample unique constituent co-expression graph generated by the facility for the example sample.

### DETAILED DESCRIPTION

The inventors have recognized limitations of conventional approaches to single-cell analysis. In particular, they have determined that being able to determine and analyze per-cell co-expression levels in a sample among large numbers of constituents would have significant value. In particular, they recognize that this would provide an improved ability to understand disease biology, perform disease diagnosis, and understand the mechanism of action of drug candidates, as a few examples.

With most single cell technologies, it is possible to treat the cells ex-vivo with immune modulators, drugs and other agents. Therefore, it is possible to study the effects of these treatments in relevant cell subsets thus providing a clearer picture of the disease biology as well as the method of action for drugs/agents. The complexity of the immune system and the desire to profile the disease biology has in practical terms meant that an ever-growing number of protein, transcriptomic and genomic markers need to be measured simultaneously at a single cell level. The capabilities of the single cell technologies have indeed come a long way over the past few years to meet this challenge. Broadly, these technologies span flow cytometry, cell imaging and sequencing technologies. Flow cytometry for instance, which relies on antibodies conjugated to fluorescent molecules to measure expression levels, has been traditionally limited to less than 15 parameters due to the limited spectral resolution and diversity of antibody conjugation. But recent advancements in spectral cytometry and non-optical methods like mass cytometry have pushed the limits to over 40 parameters per cell. In parallel, progress in cell capture technologies have created the opportunity to apply next generation sequencing (NGS) to measure RNA transcripts at the single cell level. More recently, CITE-Seq, a technique by which both protein and RNA can be measured simultaneously, has been developed. It is described by Simultaneous epitope and transcriptome measurement in single cells, Nature Methods volume 14, pages 865-868 (2017), which is hereby incorporated by reference in its entirety. In cases where the present application conflicts with a document incorporated herein by reference, the present application controls. By conjugating antibodies to unique oligonucleotides (barcodes), it is possible to perform antibody staining on cells, followed by cell capture and NGS to obtain count data for both RNA transcripts and cell bound antibody. Since, for practical purposes, an unlimited number of the oligonucleotides can be created, it is now possible to simultaneously measure expression for 10s to 100s of proteins and 1000s of transcripts for each cell in a biospecimen. Further advancement in all of these areas are currently underway and it is anticipated that these modern technologies will be adopted more broadly in research, translational science and ultimately in clinical setting. These technologies have seen growth in adoption spanning research, translational sciences and clinical settings. The number of datasets being acquired by individual labs and organizations has grown rapidly, very often exceeding tens of thousands per year.

Based on this recognition, the inventors have conceived and reduced to practice a software and/or hardware facility for analyzing per-cell co-expression of cellular constituents such as proteins and RNA transcripts (“the facility”).

In some embodiments, the facility subjects single-cell analysis instrument output data for a single subject's cell sample, or “well,” to a process—such as gating or clustering—that attributes a cell type to each cell in the sample based upon their co-expression levels of combinations of constituents that are characteristic of different cell types. As one example, in some embodiments, the facility operates to assess co-expression of proteins in tumor infiltrating cells extracted from lung cancer patients.

In some embodiments, the facility determines, for each combination of an individual cell of the sample and a constituent of interest, whether the cell has a positive expression of the constituent. In some embodiments, this involves comparing the expression level identified for the constituent in the cell by the instrument output data to a threshold expression level determined for the constituent. For example, in some embodiments, the facility determines different threshold expression levels for the constituents PD1, LAG-3, and CD103.

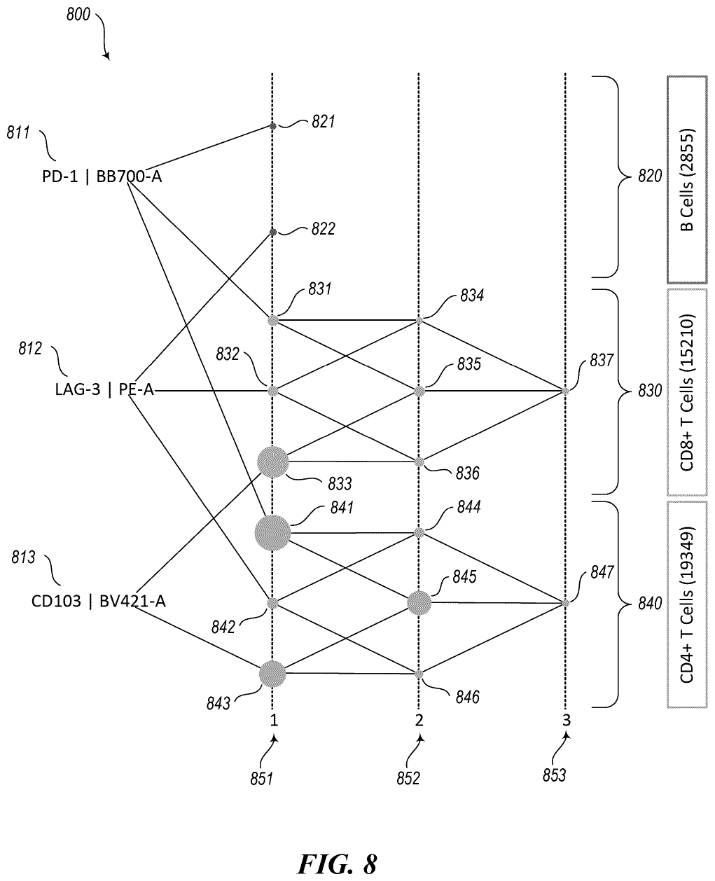

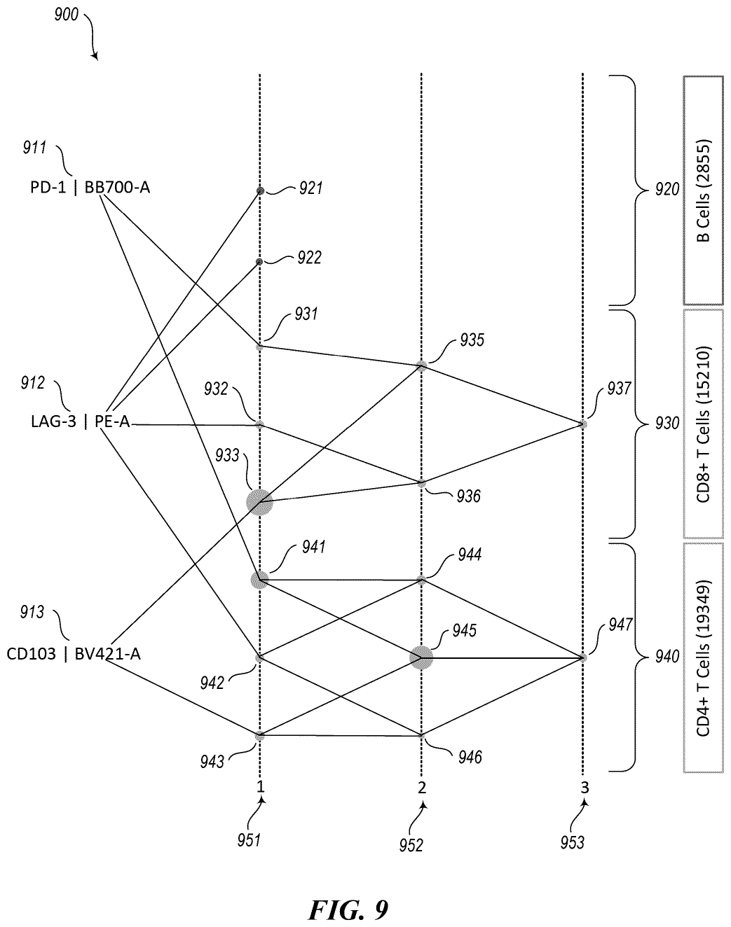

In some embodiments, the facility constructs a per-cell co-expression graph showing the relative rates at which different combinations of constituents are co-expressed within individual cells of the sample. For each constituent, the facility counts the number of cells determined to have a positive expression of the constituent, and compares it to a graph inclusion threshold. For each constituent for which the count exceeds the graph inclusion threshold, the facility adds a visual element to the graph conveying the relative magnitude of the count, such as a circular node whose diameter, area, or other size attribute reflects the relative magnitude of the count. For each combination of the constituents for which visual elements are added to the graph, the facility counts the number of cells determined to have positive expression of all of the constituents of the combination, and adds an additional visual element to the graph conveying the relative magnitude of the count. In some embodiments, the facility constructs the graph and performs the underlying analysis separately for the cells of each type. Sample graphs are shown in FIGS. 8 and 9 and discussed below.

In some embodiments, the facility persistently stores a serialized representation of the graph from which the graph can be reproduced, such as in a database together with metadata about the sample. For example, this metadata may include demographic, physiological, and/or medical data for the subject; a reference to the output data for the sample; information about the instrument that analyzed the sample, and how it was operated; etc. In some embodiments, the facility stores a compact “fingerprint” vector that it establishes to characterize the graph by applying a hashing process to the graph, or the counts used to create the graph. This fingerprint can similarly be stored in the database and linked to metadata for the sample.

In some embodiments, the facility provides a searching functionality that permits users to execute queries against the serialized representations and/or the fingerprints in the database. For example, upon detailed review of the data for a first sample among a group of samples, a user can submit a query for either (1) the most similar other samples in the group, considering all cell types, or (2) the most similar other samples in the group, considering only particular specified cell types.

In some embodiments, the facility determines similarity measures for pairs of samples using either their graphs or their fingerprints. In some embodiments, the facility uses such comparisons to cluster the samples of a group into subgroups in each of which the samples are similar.

By operating in some or all of the ways described above, the facility enables users to easily visualize constituent co-expression in a sample, search for samples having particular co-expression, and assess the similarity of pairs of samples with respect to their co-expression patterns.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by constructing much smaller and more helpful graph and fingerprint representations of a full single-cell analysis output table, the facility limits the time during which the much larger full single-cell analysis output occupies large volumes of working memory. This can also obviate the expenditure of large volumes of processing resources on ad-hoc, manually-directed analysis of the full single cell analysis output. Also, performing co-expression searching or comparison against the much more information-concentrated serialized graph and fingerprint representations demands much lower levels of data retrieval, working storage, and processing resources than performing it against full single cell analysis output tables.

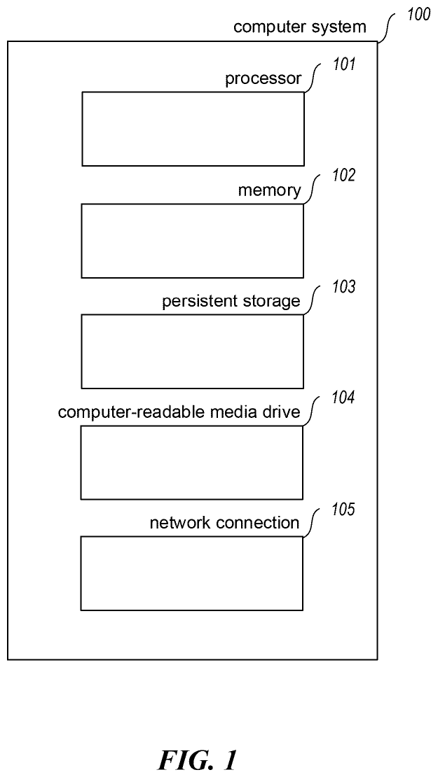

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102 for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

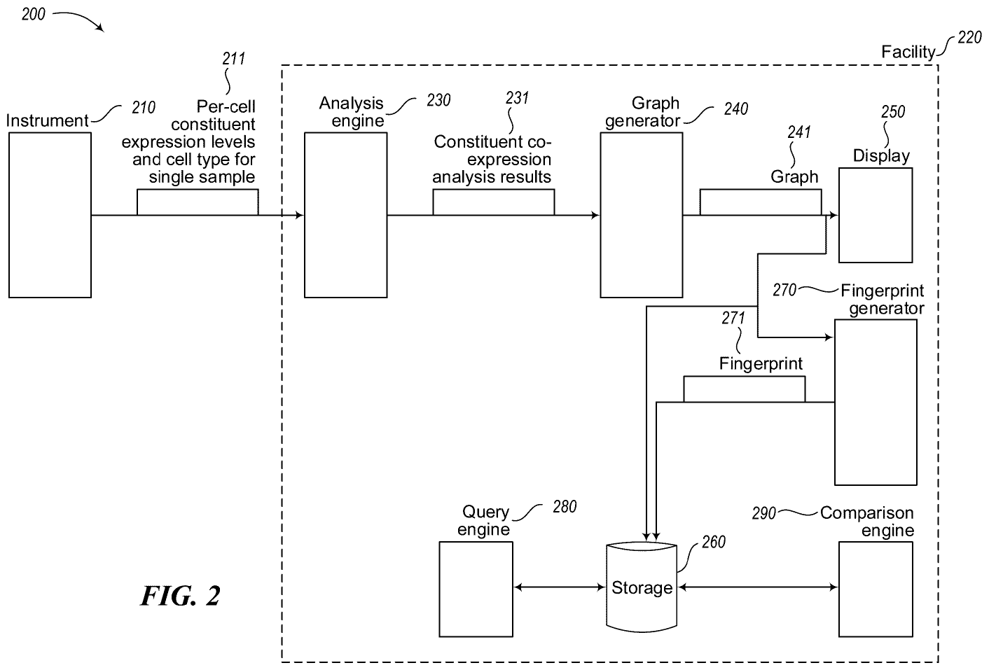

FIG. 2 is a data flow diagram showing operation of the facility in some embodiments. A single-cell analysis instrument 210 outputs data 211 relating to a single sample of cells from a particular subject. In particular, in some embodiments, this data includes, for each of the analyzed cells of the sample, the type of the cell, and constituent expression levels in the cell for each of a plurality of candidate constituents. In various embodiments, the instrument is of a variety of types, including flow cytometry instruments, mass cytometry instruments, and CITE-Seq instruments, among others. This data is received by the facility 220, and in particular an analysis engine of the facility 230. The analysis engine generates data 231 representing the results of performing constituent co-expression analysis on the instrument output data.

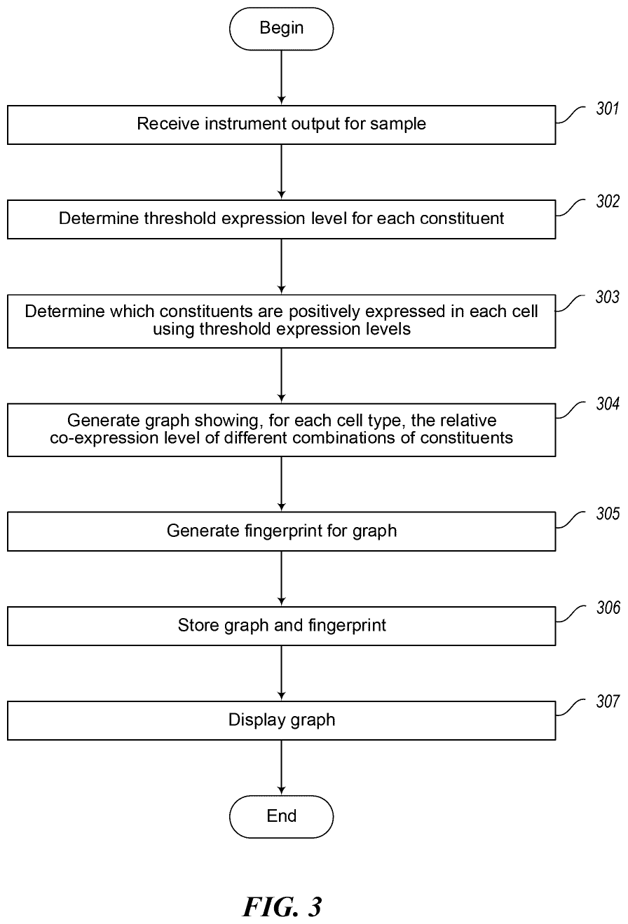

The analysis results are received by a graph generator 240 of the facility, which generates a graph 241 representing the co-expression analysis results. In some embodiments, the generated graph is received and visually presented by a display device 250. In some embodiments, the generated graph is stored persistently in a storage device 260, such as in a serialized form. In some embodiments, the graph is received by a fingerprint generator 270 of the facility. The fingerprint generator hashes the graph in order to generate a fingerprint 271 characterizing the general nature of the graph. In some embodiments, the facility stores the fingerprint 271 on the storage device. A query engine 280 of the facility receives queries from users that it processes by identifying matching graphs and/or fingerprints stored in the storage device and returning them in response to the query. A comparison engine 290 of the facility receives comparison requests to compare one or more pairs of graphs or fingerprints stored in the storage device, and score the similarity of each pair. The processing performed as part of this data flow is described in greater detail below in connection with FIG. 3.

FIG. 3 is a flow diagram showing a process performed by the facility in some embodiments to generate a constituent per-cell co-occurrence graph for a sample of cells. In act 301, the facility receives the output of a single-cell analysis instrument for a single sample of cells.

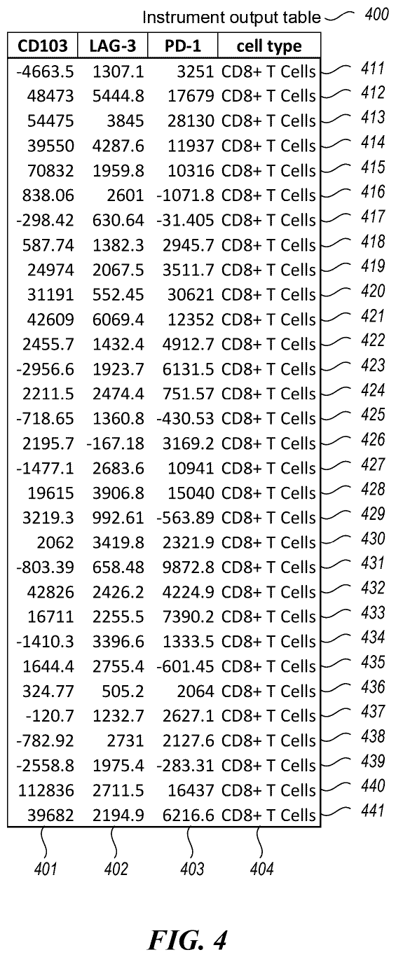

FIG. 4 is a table diagram showing sample contents of an instrument output table used by the facility in some embodiments to store instrument output data for a single sample. The instrument output table 400 is divided into rows each representing a single cell in the sample, such as rows 411-441. Each row is divided into the following columns: a CD103 column 401 containing an expression level of a CD103 constituent observed by the instrument in the cell; a LAG-3 column 402 containing an expression level of a LAG-3 constituent observed by the instrument in the cell; a PD-1 column 403 containing an expression level of a PD-1 constituent observed by the instrument in the cell; and a cell type column 404 identifying a cell type attributed to the cell by the instrument. In some embodiments, the facility uses a gating process to establish the cell type shown in column 404 based upon the level of expression of certain constituents, and/or by using a clustering approach. For example, row 413 indicates that it corresponds to a cell whose cell type is CD8+ T Cells, whose expression level for the CD103 constituent is 54475, for the LAG-3 constituent is 3845, and for the PD-1 constituent it is 28130.

While FIG. 4 and each of the table diagrams discussed below show a table whose contents and organization are designed to make them more comprehensible by a human reader, those skilled in the art will appreciate that actual data structures used by the facility to store this information may differ from the table shown, in that they, for example, may be organized in a different manner; may contain more or less information than shown; may be compressed, encrypted, and/or indexed; may contain a much larger number of rows than shown, etc.

The contents shown in the instrument output table reflect a sample of tumor infiltrating cells extracted from a lung cancer patient. These are immune cells that infiltrate tumors, are capable of interacting with the cells of a tumor, and are used in the immunotherapy approach to cancer treatment. Proteins like those listed as cellular constituents in the instrument output table are the subject of investigation for their role in this process. The inventors expect that understanding the expression of these proteins in immune cells and tumor cells—and particularly their co-expression—will provide insights into the development of new cancer treatment therapies, including those personalized to individual patients. A data set of samples including the sample shown in the contents of the instrument output table is described by Xiaoyang Wang, Maria Jaimes, Huimin Gu, Keith Shults, Santosh Putta, Vishal Sharma, Will Chow, Priya Gogoi, Kalyan Handique, and Bruce K Patterson, Cell by cell immuno- and cancer marker profiling of non-small cell lung cancer tissue: Checkpoint marker expression on CD103+, CD4+ T-cells predicts circulating tumor cells; Transl Oncol. 2021 January; 14(1): 100953, https:/www.ncbi.nlm.nih.gov/pmc/articles/PMC7683336, which is hereby incorporated by reference in its entirety.

In particular, the instrument that processed the shown sample—a Cytek Aurora flow cytometer from Cytek Biosciences Inc.—used the BV421 (brilliant violet 421) fluorophore which admits light at the 421 nanometer wavelength to measure the CD103 protein constituent and the antibody for which it has been conjugated. It uses the BB700 fluorophore (brilliant blue 700) which emits light at the 700 nanometer wavelength to measure the expression of the PD-1 constituent in cells of the sample. Further, the fluorophore PE-A phycoerythrin which emits light at 566 nanometer wavelength was used to measure the LAG-3 cellular constituent.

In some embodiments, the facility uses data generated by a variety of single-cell analysis instruments. In some embodiments, the facility uses data produced by a cytometer, such as the ZE5 cell analyzer, the S3e cell sorter, and other flow cytometry instruments from Bio-Rad; the CytoFLEX Analyzer and other cytometry products from Beckman Coulter; and Attune Nxt and CytPix and other flow cytometers from ThermoFisher Scientific, among others. In some embodiments, the facility uses data produced by a mass cytometer, such as the Helios mass cytometer, or similar products from Standard BioTools. In some embodiments, the facility uses data from sequencing instruments, from various manufacturers including those that use a droplet encapsulation technique, a microweld array technique, a combinatorial barcoding technique, or a kinetic process technique.

In various embodiments, the facility uses various techniques to select the marker agents, such as the fluorophores used in cytometry instruments and the heavy metals used by mass cytometry instruments, which exploit connections that could be made between the marked constituents and particular markers, as well as the distinguishing characteristic of the markers, such as principal wavelength for fluorophores and mass or density for heavy metal markers.

Returning to FIG. 3, in act 302, the facility determines a threshold expression level for each of the different cellular constituents as a basis of determining whether the constituent has positive expression in each cell. In various embodiments, the facility determines this threshold manually or automatically, based upon a histogram of the number of cells for which different levels of expression were measured.

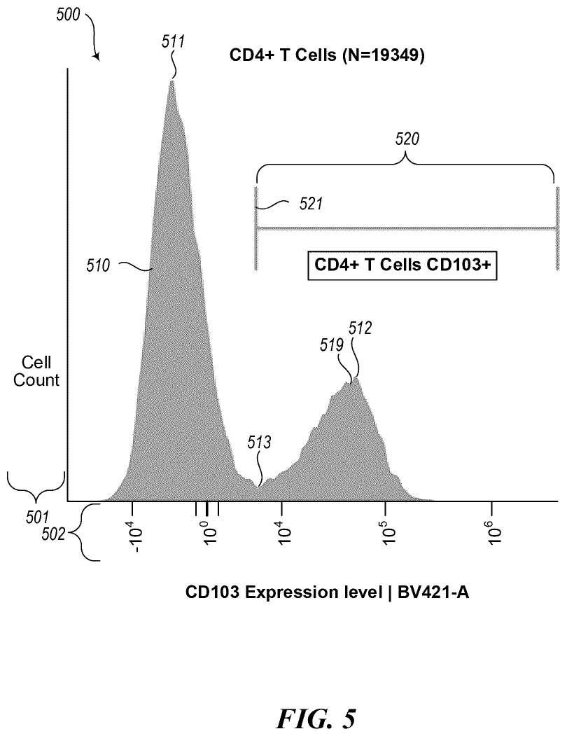

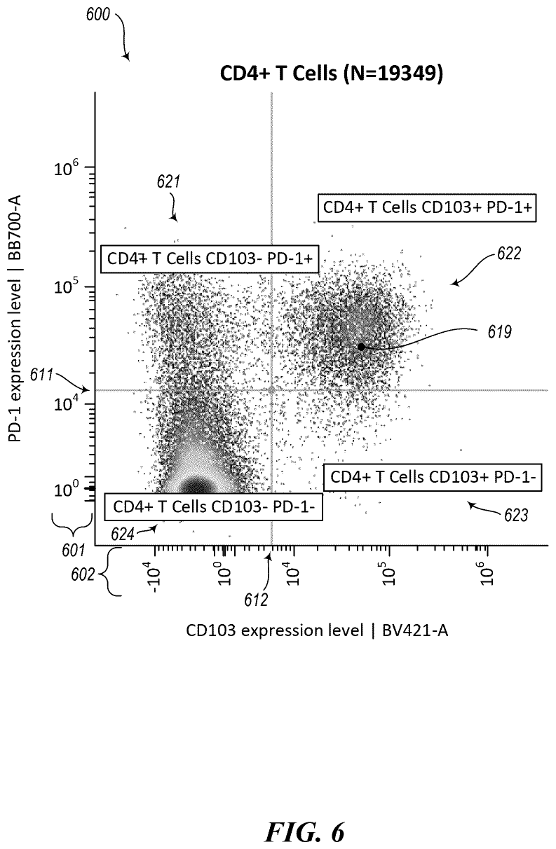

FIG. 5 is a histogram with sample contents showing the level of expression of CD4+ T Cells in the sample of the example. The graph 500 has a vertical axis 501 of cell count graphed against a horizontal axis 402 of expression level of the CD103 constituent in each cell. The horizontal axis uses linear-log scaling, which the inventors have found to be a good choice for portraying expression levels produced by flow cytometry instruments. In particular, the scaling shown on this horizontal axis—and both axes of the graph shown in FIG. 6—are linear-log scales based upon the inverse hyperbolic sine—or “arcsinh”—function described at mathworld.wolfram.com/InverseHyperbolicSine.html, which is hereby incorporated by reference in its entirety. It can be seen that the histogram has peaks 511 and 512 near the expression levels of −10² and 10⁵. It further has a trough 513 between those peaks at about the expression level 6039. Accordingly, the facility manually or automatically selects the expression level value 521 at this trough-6039—as the threshold expression level for the CD103 constituent. Thus, each of the cells of the sample shown to the right of this threshold expression level in the histogram—i.e., in range 520—is treated by the facility as having positive expression for the CD103 constituent. As an example, for the cell shown in row 413 of the instrument output table, the facility compares its CD103 expression level of 54475 to the threshold 6039 to determine that this cell has positive expression of the CD103 constituent. This is seen graphically by comparing the horizontal position of point 519 in the histogram corresponding to the same cell to the threshold expression level 521.

Proceeding in a similar manner, the facility determines the following threshold expression levels for the example's other constituents: 8853 for PD-1 and 4019 for LAG-3.

Returning to FIG. 3, in act 303, the facility determines, for each cell of the sample, which constituents are positively expressed in the cell using the threshold expression levels determined in act 302. That is, for each cells, for each constituent, the facility compares the expression level for the constituent in the cell to the threshold expression level determined for the constituent, and determines that the constituent is positively expressed in the cell if the expression level determined for the constituent in the cell exceeds the threshold expression level.

FIG. 6 is a plot diagram showing a joint expression distribution for two of the constituents in the data for the example. In particular, the plot 600 shows each cell of the sample as a single point plotted in the vertical dimension by the cell's PD-1 expression level, and in the horizontal dimension by the cell's CD103 expression level. The plot includes dividing lines 611 and 612; dividing line 611 demarcates the threshold expression level determined for the PD-1 constituent-8853, while dividing line 612 demarcates the threshold expression level determined for the CD103 constituent-6039. These dividing lines segment the positive expression for PD-1; quadrant 622, whose cells have positive expression for both PD-1 and CD103; quadrant 623 which has a positive expression level for CD103 and a negative expression level for PD-1; and quadrant 624, whose cells have negative expression for both CD103 and PD-1. Colors of the dots other than blue show increasing density of cells, making it clear that quadrant 624 is the most populous sector and quadrant 623 is the least. Continuing the example, for the cell shown in row 413 of the instrument output table, the facility compares its PD-1 expression level of 2830 to the PD-1 threshold expression level of 8853 to determine that the cell has positive expression of PD-1 constituent; and compares the cell's LAG-3 expression level of 3845 to the LAG-3 threshold level of 4019, and determines that this cell is negative of the LAG-3 constituent. This cell's positive expression for both CD103 and PD-1 is shown by the location of its representation 619 in quadrant 622 of the plot 600.

Returning to FIG. 3, in act 304, the facility generates a graph that shows, for each of the different cell types, the relative co-expression level of different combinations of constituents. In some embodiments, part of generating this graph involves counting, for each cell type, for each combination of constituents, the cells of the cell type in which co-expression of that combination of constituents occurs.

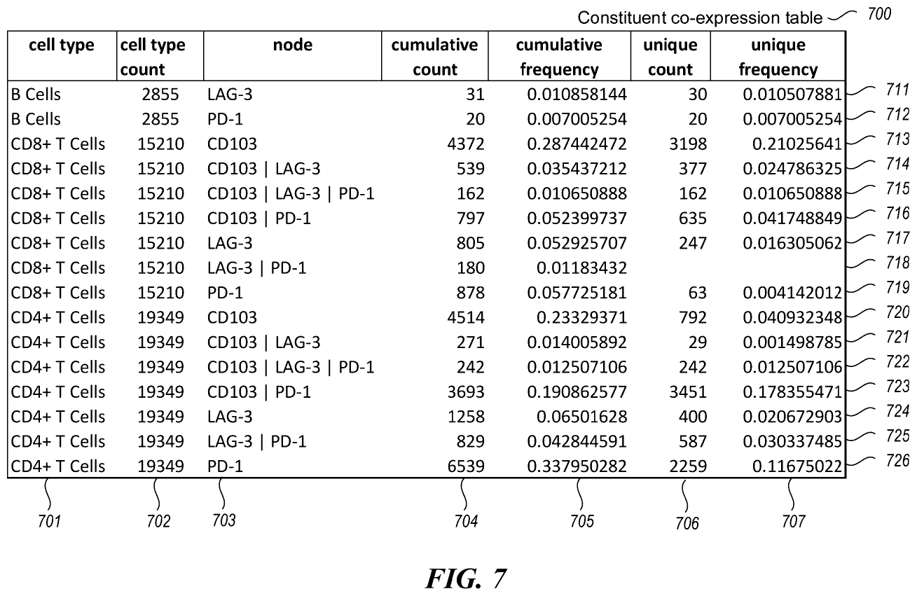

FIG. 7 is a table diagram showing sample contents of a constituent co-expression table used by the facility in some embodiments to store the constituent co-expression count for each cell type and constituent subset of a sample, on which a co-expression graph can be based. The constituent co-expression table 700 is made up of rows each corresponding to a different combination of cell type with one or more of the constituents, such as rows 711-726. Each of the rows is divided into the following columns: a cell type column 701 identifying the cell type to which the row corresponds; a cell type count column 702 showing the number of cells of the sample that were identified to be of that cell type; a node column 703 showing a combination of one or more of the constituents that are co-expressed in cells of the sample of the row's cell type; a cumulative count column 704 showing the number of cells of the row's cell type in which positive expression of each of the constituents shown in the node column occurred; a cumulative frequency column 705 showing the quotient of the cumulative count divided by the cell type count; a unique count column 706 that reduces the value in the cumulative count column by the number in which a proper superset of the constituents in the node column occur; and a unique frequency column 707 showing the quotient of the value in the unique count column divided by the value in the cell type count column. For example, row 713 indicates that, of the 15,210 CD8+ T Cells in the sample, 4,372 of them were found to have positive expression of the CD103 constituent, which amounts to 28.7442472% of the CD8+ T Cells. Further, of these 4,372 or 3,198 failed to have positive expression of any other constituent, which is 21.025641% of all the CD8+ T Cells. In some embodiments—as shown—the facility excludes from the constituent co-expression table rows for which the cumulative account unique count is zero, or below a non-zero inclusion threshold.

To further extend the example with respect to the cell shown row 413 of the instrument output table, this cell is represented in the unique count for row 716, which includes only cells which are positive for CD103 and PD-1, and negative for the other constituent, LAG-3. This cell is also included in the cumulative counts for rows 713, 716, and 719 because of this cell's expression positivity for CD103 and PD-1.

FIGS. 8 and 9 show two co-expression graphs generated by the facility for the example sample. FIG. 8 shows a sample cumulative co-expression graph generated by the facility for the example sample. The graph 800 is divided into subgraphs 820, 830, and 840 each corresponding to one of the three cell types in the sample. For example, subgraph 830 corresponds to the CD8+ T cell type. Points 811-813 along the left side of the graph each identify a different one of the constituents whose expression levels are measured in the sample. For example, point 812 identifies the LAG-3 constituent. The graph is organized into levels to the right of the constituents: level 1 851, level 2 852, and level 3 853. Level 1 contains nodes 821, 822, 831, 832, 833, 841, 842, and 843, each of which represents the cumulative expression level for a different combination of a cell type with a single constituent. For example, node 831 represents all of the CD8+ T Cells of the sample found to have positive expression of the PD-1 constituent, including the cell shown in row 413 of the instrument output table. Node 833 represents cumulatively the CD8+ T Cells of the sample found to have positive expression of the CD103 constituent, including the cell shown in row 413 of the instrument output table. Nodes at the second level of the graph represent cells of a particular cell type found to have positive expression of at least two different constituents. The nodes at the second level are nodes 834, 835, 836, 844, 845, and 846. For example, node 835 represents the CD8+ T Cells of the sample found to have positive expression of both the PD-1 and CD103 constituents, including the cell shown in row 413 of the instrument output table. The third level of the graph contains nodes representing cells of a particular cell type in which positive expression of all three constituents were found. These include nodes 837 and 847. For example, node 837 represents the CD8+ T Cells of the sample in which positive expression of all of the three measured constituent were found: PD-1, LAG-3, and CD103. The size of each node—such as its diameter, its area, etc.—reflects the number of cells of the sample represented by the node. For example, node 831 represents 878 cells of the sample (found at the intersection of row 719 and column 704 of the constituent co-expression table). Node 833, which is larger, represents 4,372 cells of the sample (found at the intersection of row 713 and column 704 of the constituent co-expression table). The designation of the graph shown in FIG. 8 as cumulative means that each cell is represented in all of the nodes for whose constituents the cell has positive expression. For example, all of the 797 cells represented by node 835 (including the cell shown in row 413 of the instrument output table), because they have positive expression of the both the PD-1 constituent and the CD103 constituent, are also represented in node 831 for the PD-1 constituent and node 833 for the CD103 constituent. In some embodiments, nodes are only included in graphs generated by the facility if they qualify for inclusion in the constituent co-expression table, such as by meeting minimum cell count thresholds.

FIG. 9 is a sample unique constituent co-expression graph generated by the facility for the example sample. The unique co-expression graph 900 is organized in a manner similar to the cumulative co-expression graph 800, in that each node is connected to one or more of the constituents 911-913. The size of many of the nodes differ between the two graphs, however, and one node (834) present in the cumulative co-expression is absent from the unique co-expression graph as a result of its reduced size there. Fundamentally, the unique co-expression graph differs from the cumulative co-expression graph in that each cell is represented only in a single node, corresponding to all of the constituents whose positive expression was found in the cell. For example, none of the 635 CD8+ T Cells represented by node 935 (found at the intersection of row 716 and column 706 of the constituent co-expression table)—including the cell shown in row 413 of the instrument output table—is among the 63 cells represented by node 931 (found at the intersection of row 719 and column 706 of the constituent co-expression table) or the 3,198 cells represented by node 933 (found at the intersection of row 713 and column 706 of the constituent co-expression table). Thus, in the unique co-expression graph each cell is represented only in the node corresponding to the full set of constituents having positive expression in it.

Returning to FIG. 3, in act 305, the facility generates a fingerprint representing the contents of the graph. In some embodiments, the facility instead or additionally generates a separate fingerprint for each of the graph's subgraphs. In various embodiments, the facility uses a variety of approaches to generate these fingerprints.

Each fingerprint (in some embodiments, constituting binary or real values) is a vector of pre-specified number of entries (bits or real numbers) providing a convenient representation of the graph. Typically, a fingerprint is a one-way transformation of a graph to a vector; i.e., the fingerprint does not contain adequate information to derive the graph back from a fingerprint. Depending on the characteristics of the graph that one desires to capture in a fingerprint, there are several computational methods to derive a fingerprint from a graph. In some embodiments, the fingerprint generated by the facility reflects co-expression patterns in the graph; such as on the sub-graphs derived from a graph. Generally—including the cell shown in row 413 of the instrument output table—speaking, in some embodiments, an objective is to have the same entry (e.g., 20th bit) in two fingerprints turned-on (if binary), or have similar real values for the same or similar sub-graph (e.g., CD103, PD-1, CD103|PD-1 in CD4+ T Cells). In some embodiments, the facility accomplishes this by:
1. Enumerate all possible sub-graphs containing up to K nodes.
2. Compute a hash for each sub-graph by applying hash function, such as in python, to an adjacency matrix representing the connections between nodes.
3. Compute fingerprint entry id as the remainder obtained when the hash is divided by the number of entries in the fingerprint, such as 1024).
4. For real-valued fingerprints, this entry, instead of being set to 1, is set to the sum of frequencies observed at each of the sub-graphs to simultaneously capture the size (weight) of nodes in a sub-graph.

Following the above procedure, multiple sub-graphs may map to the same entry in the fingerprint. However, two graphs with the same sub-graphs would result in the same entry being set. In some embodiments, the facility uses one or more of the fingerprinting methods described in David Rogers and Mathew Hahn, Extended-Connectivity Fingerprints, J. Chem. Inf. Model. 2010, 50, 5, 742-754; Raymond E. Carhart, Dennis H. Smith, and R. Venkataraghavan, Atom pairs as molecular features in structure-activity studies: definition and applications, J. Chem. Inf. Comput. Sci. 1985, 25, 2; and B Zagidullin, Z Wang, Y Guan, E Pitkänen, J Tang, Comparative analysis of molecular fingerprints in prediction of drug combination effects, Briefings in Bioinformatics, Volume 22, Issue 6, November 2021, doi.org/10.1093/bib/bbab291, each of which is hereby incorporated by reference in its entirety.

Further, with the advent of Convolutional Neural Networks (and Graph Neural Networks), in the recent years, it is possible to encode a graph as fingerprint using a multi-layer neural network such as is described by Duvenaud, D., et al. Convolutional Networks on Graphs for Learning Molecular Fingerprints. The 28th International Conference on Neural Information Processing Systems. 2018.12, which is hereby incorporated by reference in its entirety. Broadly speaking, each layer of the neural network is transmitting a small amount of information from one node to another via the connections, effectively modeling the underlying structure of the graph. Each of the documents identified in this paragraph is hereby incorporated by reference in its entirety.

Once each graph (or a sub-graph) has been modeled as a fingerprint, it is a very convenient form to store in the database and search. Note that more than one type of fingerprint can be stored in the database to benefit different use cases. For example, sub-graph based fingerprints described above are very convenient to search for similar co-expression patterns between two datasets. More specifically such queries can also be made on a sub-pattern; e.g., find all datasets in the database that have a similar co-expression pattern for PD-1, CD103, LAG-3 in CD4+ T Cells while ignore the patterns in B Cells and NK Cells.

In act 306, the facility persistently stores the graph generated in act 304 and the fingerprint(s) generated in act 305. In act 307, the facility causes the graph generated in act 304 to be displayed for review and exploration by a user. After act 307, this process concludes.

Those skilled in the art will appreciate that the acts shown in FIG. 3 and in each of the flow diagrams discussed below may be altered in a variety of ways. For example, the order of the acts may be rearranged; some acts may be performed in parallel; shown acts may be omitted, or other acts may be included; a shown act may be divided into sub-acts, or multiple shown acts may be combined into a single act, etc.

As described above, once the graph and fingerprint are stored by the facility, they can be used in order to service search queries for graphs. In some embodiments, in order to specify a search query, a user selects the graphs generated by the facility for one or more particular samples, and requests that graphs be returned for other samples that are similar. In some embodiments, the same type of searching is available for subgraphs selected by the user. In some embodiments, specifying a graph search query involves specifying particular attributes of the graph, such as those that show a co-expression level of a certain specified group of constituents among cells of a certain type. In some embodiments, part of the search query includes metadata attributes specified for a graph, a sample, or the subject from which or whom the sample was extracted. These attributes can span a wide variety, including a range of dates when the sample was extracted or analyzed; the instrument type or particular instrument used to do the analysis; and the size of the sample; details of the subject, such as age, sex, ethnic group, diagnosed pathologies, previous procedures, height, weight, resting heart rate, blood pressure, body mass index, medicines or other therapies, test results, etc. In some embodiments, the graphs or subgraphs returned by a query are displayed; stored separately; flagged for later review; etc.

In some embodiments, the facility supports the comparison of pairs of graphs or subgraphs to produce similarity scores. A number measures of similarity that are biologically meaningful are possible based on this graphical representation between two single cell datasets based on this graph representation. Broadly speaking, two graphs are considered more similar to each other if more of the nodes are similar to each other. In other words, graph similarity is measured as an aggregation across the nodes in the graphs being compared. In some embodiments, the facility determines a Jaccard similarity metric between two nodes as a measure of their similarity:

$$σ = \frac{1}{N} {\sum }_{i = 1}^{c} {w}_{i} {\sum }_{j = 1}^{n} \frac{min ( {f}_{ij}^{a} , {f}_{ij}^{b} )}{max ( {f}_{ij}^{a} , {f}_{ij}^{b} )}$$

where
ƒᵢⱼ^{a} is the number of cell in node j as a fraction of number of cell in cell type i in sample a;
ƒᵢⱼ^{b} is the number of cell in node j as a fraction of number of cell in cell type i in sample b;
wᵢ is a weight factor for cell type i;
N is the total number of nodes across the two graphs

In some embodiments, this approach is modified in a variety of ways, to create similarity metrics that are appropriate for different applications. For instance, in some embodiments:
The summation is limited to specific cell type/s (e.g. only T Cells), in other words, set weight for specific cell types to zero, to focus only on cell types that relevant to a specific disease area or application.
The summation is limited to nodes with a minimum number of cells.
Alternative functional forms are used to measure similarity between two nodes instead of Jaccard similarity. For instance, the difference in number of cells as a fraction of parent cell type can be blunted by thresholding the maximum values for the fractions ƒᵢⱼ^{a} and ƒᵢⱼ^{b} to stress on the presence of a minimal frequency of the cells of certain types (nodes) rather than the quantity.
Nodes are merged together first before evaluating similarity. This would allow wild card searching; for example a node that is T Cells with PD1+CTLA+ could be considered similar to T Cells with PD1+TIM-3+.

In various embodiments, the facility generates these similarity scores using one or more of the techniques described in Peter Wills, François G. Meyer. Metrics for graph comparison: A practitioner's guide, Feb. 12, 2020, https://doi.org/10.1371/journal.pone.0228728; G. Jeh and J. Widom. “SimRank: a measure of structural-context similarity”, In KDD'02: Proceedings of the Eighth ACM SIGKDD International Conference on Knowledge Discovery and Data Mining, pp. 538-543. ACM Press, 2002; and Cook D J H LB, editor. Mining Graph Data. Wiley; 2006, each of which is hereby incorporated by reference in its entirety. In some embodiments, the facility automatically performs these comparisons exhaustively or semi-exhaustively across all pairs of graphs or fingerprints contained in a selected set of graphs or fingerprints, and uses the matrix of similarity scores to automatically cluster the graphs or fingerprints into groups of similar graphs or fingerprints, connoting groups of similar examples.

In some embodiments, the facility uses the graphs and/or fingerprints it generates as a basis for performing unsupervised machine learning, such as via clustering.

In some embodiments, the facility uses the graphs and/or fingerprints that it generates as a basis for performing supervised machine learning, in which machine learning models are constructed and trained to predict a value of a dependent variable based upon the graphs and/or fingerprints as dependent variable values. For example, in some embodiments, the facility trains and applies such machine learning models in order to predict values of any of the following dependent variables for the subject from which a particular sample was located: disease state; response to particular therapy; in vivo disease progression; ex vivo disease progression; and tumor infiltration, among others.

In some embodiments, rather than using a binary determination of constituent positivity that is based on a single expression threshold for each constituent, the facility uses a probabilistic definition of positivity, where a cell with a low expression level for a particular constituent is counted with a lower weight than a cell with a higher expression level of the same constituent in the counts made by the facility to constitute the size of nodes of the co-expression graph.

The following are included among the facility's embodiments:
1. A method in a computing system for generating a graph, comprising:
   accessing a data object emitted by a single-cell analysis instrument with respect to a sample, the data object indicating, for each of a plurality of cells within the sample, for each of a plurality of cellular constituents, an expression level determined by the instrument for the constituent in the cell;
   initializing the graph; and
   populating the initialized graph, by:
      for each of the plurality of constituents:
         for each of the plurality of cells:
         determining whether the cell has positive expression of the constituent by comparing the data object's indication of the expression level of the constituent in the cell to a positive expression threshold;
         where it is determined that the cell has positive expression of the constituent, storing an indication that the cell has positive expression of the constituent;
         counting the number of stored indications that cells have positive expression of the constituent to obtain a count;
         setting an individual constituent graph inclusion flag for the constituent to either true or false in accordance with a comparison of the count to a graph inclusion threshold;
      for each of the plurality of constituents whose individual constituent graph inclusion flags are set:
         adding to the graph a node corresponding to the constituent whose appearance reflects the count obtained for the constituent;
      or each of a plurality of different combinations of constituents whose individual constituent graph inclusion flags are set to true:
         counting the number of cells for each of which indications are stored that the cell has positive expression of all of the constituents of the combination; and
         adding to the graph a node corresponding to the combination whose appearance reflects the count obtained for the combination.
2. The method of embodiment 1 wherein the plurality of cellular constituents are selected from among transcriptomic cellular constituents, proteomic cellular constituents, and genomic cellular constituents.
3. The method of embodiment 1 or embodiment 2, the method further comprising:
   applying a hashing technique to data representing the generated graph to obtain a co-expression fingerprint for the sample characterizing the generated graph; and
   persistently storing the obtained fingerprint.
4. The method of embodiment 1 or embodiment 2 for each of the plurality of cells:
   determining a cell type of the cell from among a multiplicity of cell types,
      wherein the populating is performed separately for the cells determined to be of each of a plurality of cell types selected from among the multiplicity of subtypes, such that the generated graph contains a distinct subgraph for each of the selected cell types.
5. The method of embodiment 4, the method further comprising:
   performing the accessing, initializing, populating, and storing twice, once for a first data object corresponding to a first sample, and once for a second data object corresponding to a second sample different from the first sample, to obtain first and second graphs; and
   receiving user input designating one of the selected cell types; and
   determining a quantitative similarity measure between the first and second graphs representing a level of similarity between co-expression patterns in the first and second samples with respect to the designated cell type, using data representing the first and second graphs.
6. The method of embodiment 4 or embodiment 5, the method further comprising:
   for each of a plurality of samples, performing the accessing, initializing, populating, and storing to obtain a graph for the sample; and
   for a distinguished one of the plurality of selected cell types:
   applying a clustering technique to the subgraph of the obtained graphs for the distinguished cell type to organize samples among the plurality of samples into a plurality of clusters, each of the clusters containing samples whose graph subgraphs for the distinguished cell type reflect similar co-expression patterns.
7. The method of any one of embodiments 4-6, further comprising: for a distinguished one of the plurality of selected cell types:
   applying a hashing technique to data representing the subgraph of the generated graph for the distinguished cell type to obtain a co-expression fingerprint for the sample characterizing the subgraph; and
   persistently storing the obtained fingerprint.
8. The method of embodiment 3 or 7, the method further comprising:
   repeating the accessing, initializing, populating, applying, and storing for a plurality of data objects each corresponding to a different sample to obtain both a generated graph and a co-expression fingerprint for each of the plurality of data objects;
   receiving a query specifying a co-expression pattern identifying at least two constituents;
   selecting a proper subset of the stored co-expression fingerprint that match the co-expression pattern specified by the query; and
   for each of at least a portion of the selected stored co-expression fingerprints, outputting information about the corresponding generated graph.
9. The method of embodiment 3 or 7, the method further comprising:
   repeating the accessing, initializing, populating, applying, and storing for a plurality of data objects each corresponding to a different sample to obtain both a generated graph and a co-expression fingerprint for each of the plurality of data objects;
   for each of the plurality of data objects:
   accessing a conclusion reached with respect to the sample to which the data object corresponds or a subject from which the sample was obtained;
   constructing a training observation in which the co-expression fingerprint generated for the data object is an independent variable value, and the accessed conclusion is a dependent variable value; and
using the constructed training observations to train a machine learning model to infer conclusion from co-expression fingerprint for an additional data object.
10. The method of embodiment 1 or embodiment 2, the method further comprising:
   performing the accessing, initializing, populating, and storing twice, once for a first data object corresponding to a first sample, and once for a second data object corresponding to a second sample different from the first sample, to obtain first and second graphs; and
   determining a quantitative similarity measure between the first and second graphs representing a level of similarity between co-expression patterns in the first and second samples, using data representing the first and second graphs.
11. The method of embodiment 1 or embodiment 2, the method further comprising:
   for each of a plurality of samples, performing the accessing, initializing, populating, and storing to obtain a graph for the sample; and
   applying a clustering technique to the obtained graphs to organize samples among the plurality of samples into a plurality of clusters, each of the clusters containing samples whose graphs reflect similar co-expression patterns.
12. The method of any one of embodiments 1-11, the method further comprising causing the populated graph to be presented on a dynamic display device.
13. The method of any one of embodiments 1-12, the method further comprising causing the populated graph to be persistently stored.
14. The method of embodiment 13, the method further comprising:
   repeating the accessing, initializing, populating, and storing for a plurality of data objects each corresponding to a different sample to obtain a stored graph for each of the plurality of data objects;
   receiving a query specifying a co-expression pattern identifying at least two constituents;
   selecting a proper subset of the stored graphs that match the co-expression pattern specified by the query; and
   for each of at least a portion of the selected stored graphs, outputting information about the graph.
15. The method of embodiment 14 wherein the outputted information comprises at least one of (1) the stored graph and (2) information about the sample from whose data object the graph was generated.
16. One or more computer memories collectively storing a data structure with respect to a sample comprising a plurality of animal cells, the data structure comprising:
   first data elements each representing one of a plurality of first-degree nodes, each of the first-degree nodes corresponding to a different one of a plurality of cellular constituents, each first data element comprising a quantitative indication of the portion of cells of the sample in which the constituent has positive expression; and
   second data elements each representing one of a plurality of greater-than-first-degree nodes, each of the greater-than-first-degree degree nodes corresponding to a different subset of the plurality of constituents containing at least two of the plurality of constituents, each second data element comprising a quantitative indication of the portion of cells of the sample in which the subset of constituents all have positive expression,
      such that the contents of the data structure are usable to generate a visual co-expression graph characterizing the sample.
17. The one or more computer memories of embodiment 16 wherein a cell type is attributed to each of the plurality of cells, and wherein the data structure comprises a set of first and second data elements for each of a plurality of different cell types.
18. The one or more computer memories of embodiment 16 or 17 wherein, for each of the second data elements, the second data element further comprises a connected node list identifying two or more nodes other than the node that the second data element represents, wherein each identified node corresponds to a subset of the plurality of constituents that is also a subset of the subset of the plurality of constituents to which the node represented by the second data element corresponds,
   such that the contents of the data structure further usable to include in the generated visual co-expression graph, for each of the second data elements, edges between (1) the node represented by the second data element and (2) the nodes identified by the connected node list in the second data element.
19. The one or more computer memories of any of embodiments 16-18 wherein the first and second data elements comprise a serialized representation of the co-expression graph.
20. The one or more computer memories of any of embodiments 16-19 wherein the data structure further comprises:
   a third data element hashed from the first and second data elements to characterize the sample.
21. The one or more computer memories of any of embodiments 16-20 wherein the data structure comprises first and second data elements for each of a plurality of different samples,
and wherein the data structure further comprises:
   a fourth data element constituting a search index that, for each of a plurality of co-expression pattern characterizations, maps from the co-expression pattern characterization to the first and second data elements for samples among the plurality of samples that match the co-expression pattern characterization, such that the contents of the data structure are further usable to service queries for samples that each specify a particular co-expression pattern characterization.
   22. One or more instances of computer-readable media collectively having contents configured to cause a computing system to perform a method for generating a graph, the method comprising:
      accessing a data object emitted by a single-cell analysis instrument with respect to a sample, the data object indicating, for each of a plurality of cells within the sample, for each of a plurality of cellular constituents, an expression level determined by the instrument for the constituent in the cell;
      initializing the graph; and
      populating the initialized graph, by:
         for each of the plurality of constituents:
            for each of the plurality of cells:
            determining a positive expression metric indicating the extent to which the cell has positive expression of the constituent by comparing the data object's indication of the expression level of the constituent in the cell to a positive expression baseline;
            based on the positive expression metrics determined for the constituent for the cells of the plurality, determining an expression level for the constituent for the cells of the plurality;
            setting an individual constituent graph inclusion flag for the constituent to either true or false on the basis of the expression level determined level for the constituent for the cells of the plurality;
         for each of the plurality of constituents whose individual constituent graph inclusion flags are set:
            adding to the graph a visual element corresponding to the constituent whose appearance reflects the expression level determined level for the constituent for the cells of the plurality;
         for each of a plurality of different combinations of constituents whose individual constituent graph inclusion flags are set to true:
            based on the positive expression metrics determined for the constituents of the combination for the cells of the plurality, determining an expression level for the constituents of the combination for the cells of the plurality; and
            adding to the graph a visual element corresponding to the combination whose appearance reflects the expression level determined level for the constituents of the combination for the cells of the plurality.

23. The one or more instances of computer-readable media of embodiment 22, wherein the method further comprises the method of any of embodiments 3-15.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system for generating a graph, comprising:
accessing a data object emitted by a single-cell analysis instrument with respect to a sample, the data object indicating, for each of a plurality of cells within the sample, for each of a plurality of cellular constituents, an expression level determined by the instrument for the constituent in the cell;
initializing the graph; and
populating the initialized graph, by:
for each of the plurality of constituents:
for each of the plurality of cells:
determining whether the cell has positive expression of the constituent by comparing the data object's indication of the expression level of the constituent in the cell to a positive expression threshold;
where it is determined that the cell has positive expression of the constituent, storing an indication that the cell has positive expression of the constituent;
counting the number of stored indications that cells have positive expression of the constituent to obtain a count;
setting an individual constituent graph inclusion flag for the constituent to either true or false in accordance with a comparison of the count to a graph inclusion threshold;
for each of the plurality of constituents whose individual constituent graph inclusion flags are set:
adding to the graph a node corresponding to the constituent whose appearance reflects the count obtained for the constituent;
or each of a plurality of different combinations of constituents whose individual constituent graph inclusion flags are set to true:
counting the number of cells for each of which indications are stored that the cell has positive expression of all of the constituents of the combination; and
adding to the graph a node corresponding to the combination whose appearance reflects the count obtained for the combination.

2. The method of claim 1 wherein the plurality of cellular constituents are selected from among transcriptomic cellular constituents, proteomic cellular constituents, and genomic cellular constituents.

3. The method of claim 1, the method further comprising:
applying a hashing technique to data representing the generated graph to obtain a co-expression fingerprint for the sample characterizing the generated graph; and
persistently storing the obtained fingerprint.

4. The method of claim 1 for each of the plurality of cells:
determining a cell type of the cell from among a multiplicity of cell types, wherein the populating is performed separately for the cells determined to be of each of a plurality of cell types selected from among the multiplicity of subtypes, such that the generated graph contains a distinct subgraph for each of the selected cell types.

5. The method of claim 4, the method further comprising:
performing the accessing, initializing, populating, and storing twice, once for a first data object corresponding to a first sample, and once for a second data object corresponding to a second sample different from the first sample, to obtain first and second graphs; and
receiving user input designating one of the selected cell types; and
determining a quantitative similarity measure between the first and second graphs representing a level of similarity between co-expression patterns in the first and second samples with respect to the designated cell type, using data representing the first and second graphs.

6. The method of claim 4, the method further comprising:
for each of a plurality of samples, performing the accessing, initializing, populating, and storing to obtain a graph for the sample; and
for a distinguished one of the plurality of selected cell types:
applying a clustering technique to the subgraph of the obtained graphs for the distinguished cell type to organize samples among the plurality of samples into a plurality of clusters, each of the clusters containing samples whose graph subgraphs for the distinguished cell type reflect similar co-expression patterns.

7. The method of claim 4, further comprising:
for a distinguished one of the plurality of selected cell types:
applying a hashing technique to data representing the subgraph of the generated graph for the distinguished cell type to obtain a co-expression fingerprint for the sample characterizing the subgraph; and
persistently storing the obtained fingerprint.

8. The method of claim 3, the method further comprising:
repeating the accessing, initializing, populating, applying, and storing for a plurality of data objects each corresponding to a different sample to obtain both a generated graph and a co-expression fingerprint for each of the plurality of data objects;
receiving a query specifying a co-expression pattern identifying at least two constituents;
selecting a proper subset of the stored co-expression fingerprint that match the co-expression pattern specified by the query; and
for each of at least a portion of the selected stored co-expression fingerprints, outputting information about the corresponding generated graph.

9. The method of claim 3, the method further comprising:
repeating the accessing, initializing, populating, applying, and storing for a plurality of data objects each corresponding to a different sample to obtain both a generated graph and a co-expression fingerprint for each of the plurality of data objects;
for each of the plurality of data objects:
accessing a conclusion reached with respect to the sample to which the data object corresponds or a subject from which the sample was obtained;
constructing a training observation in which the co-expression fingerprint generated for the data object is an independent variable value, and the accessed conclusion is a dependent variable value; and
using the constructed training observations to train a machine learning model to infer conclusion from co-expression fingerprint for an additional data object.

10. The method of claim 1, the method further comprising:
performing the accessing, initializing, populating, and storing twice, once for a first data object corresponding to a first sample, and once for a second data object corresponding to a second sample different from the first sample, to obtain first and second graphs; and
determining a quantitative similarity measure between the first and second graphs representing a level of similarity between co-expression patterns in the first and second samples, using data representing the first and second graphs.

11. The method of claim 1, the method further comprising:
for each of a plurality of samples, performing the accessing, initializing, populating, and storing to obtain a graph for the sample; and
applying a clustering technique to the obtained graphs to organize samples among the plurality of samples into a plurality of clusters, each of the clusters containing samples whose graphs reflect similar co-expression patterns.

12. The method of claim 1, the method further comprising causing the populated graph to be presented on a dynamic display device.

13. The method of claim 1, the method further comprising causing the populated graph to be persistently stored.

14. The method of claim 13, the method further comprising:
repeating the accessing, initializing, populating, and storing for a plurality of data objects each corresponding to a different sample to obtain a stored graph for each of the plurality of data objects;
receiving a query specifying a co-expression pattern identifying at least two constituents;
selecting a proper subset of the stored graphs that match the co-expression pattern specified by the query; and
for each of at least a portion of the selected stored graphs, outputting information about the graph.

15. The method of claim 14 wherein the outputted information comprises at least one of (1) the stored graph and (2) information about the sample from whose data object the graph was generated.

16. One or more computer memories collectively storing a data structure with respect to a sample comprising a plurality of animal cells, the data structure comprising:
first data elements each representing one of a plurality of first-degree nodes, each of the first-degree nodes corresponding to a different one of a plurality of cellular constituents, each first data element comprising a quantitative indication of the portion of cells of the sample in which the constituent has positive expression; and
second data elements each representing one of a plurality of greater-than-first-degree nodes, each of the greater-than-first-degree degree nodes corresponding to a different subset of the plurality of constituents containing at least two of the plurality of constituents, each second data element comprising a quantitative indication of the portion of cells of the sample in which the subset of constituents all have positive expression,
such that the contents of the data structure are usable to generate a visual co-expression graph characterizing the sample.

17. The one or more computer memories of claim 16 wherein a cell type is attributed to each of the plurality of cells,
and wherein the data structure comprises a set of first and second data elements for each of a plurality of different cell types.

18. The one or more computer memories of claim 16 wherein, for each of the second data elements, the second data element further comprises a connected node list identifying two or more nodes other than the node that the second data element represents, wherein each identified node corresponds to a subset of the plurality of constituents that is also a subset of the subset of the plurality of constituents to which the node represented by the second data element corresponds,
such that the contents of the data structure further usable to include in the generated visual co-expression graph, for each of the second data elements, edges between (1) the node represented by the second data element and (2) the nodes identified by the connected node list in the second data element.

19. The one or more computer memories of claim 16 wherein the first and second data elements comprise a serialized representation of the co-expression graph.

20. The one or more computer memories of claim 16 wherein the data structure further comprises:
a third data element hashed from the first and second data elements to characterize the sample.

21. The one or more computer memories of claim 16 wherein the data structure comprises first and second data elements for each of a plurality of different samples,
and wherein the data structure further comprises:
a fourth data element constituting a search index that, for each of a plurality of co-expression pattern characterizations, maps from the co-expression pattern characterization to the first and second data elements for samples among the plurality of samples that match the co-expression pattern characterization,
such that the contents of the data structure are further usable to service queries for samples that each specify a particular co-expression pattern characterization.

22. One or more instances of computer-readable media collectively having contents configured to cause a computing system to perform a method for generating a graph, the method comprising:
accessing a data object emitted by a single-cell analysis instrument with respect to a sample, the data object indicating, for each of a plurality of cells within the sample, for each of a plurality of cellular constituents, an expression level determined by the instrument for the constituent in the cell;
initializing the graph; and
populating the initialized graph, by:
for each of the plurality of constituents:
for each of the plurality of cells:
determining a positive expression metric indicating the extent to which the cell has positive expression of the constituent by comparing the data object's indication of the expression level of the constituent in the cell to a positive expression baseline;
based on the positive expression metrics determined for the constituent for the cells of the plurality, determining an expression level for the constituent for the cells of the plurality;
setting an individual constituent graph inclusion flag for the constituent to either true or false on the basis of the expression level determined level for the constituent for the cells of the plurality;
for each of the plurality of constituents whose individual constituent graph inclusion flags are set:
adding to the graph a visual element corresponding to the constituent whose appearance reflects the expression level determined level for the constituent for the cells of the plurality;
for each of a plurality of different combinations of constituents whose individual constituent graph inclusion flags are set to true:
based on the positive expression metrics determined for the constituents of the combination for the cells of the plurality, determining an expression level for the constituents of the combination for the cells of the plurality; and
adding to the graph a visual element corresponding to the combination whose appearance reflects the expression level determined level for the constituents of the combination for the cells of the plurality.

23. (canceled)
